(19) United States
(12) Patent Application Publication
(10) Pub. No.: US 2005/0192242 A1
(43) Pub. Date: Sep. 1, 2005

(54) Therapeutic fusion protein transgenes

(76) Inventors: Zane C. Neal, Cambria, WI (US); Hans Herweijer, Madison, WI (US); Jon A. Wolff, Madison, WI (US)

Correspondence Address: MIRUS CORPORATION, 505 SOUTH ROSA RD, MADISON, WI, 53719, US

(21) Appl. No.: 11/095,793

(22) Filed: Mar. 31, 2005

Related U.S. Application Data
(63) Continuation-in-part of application No. 10/600,290, filed on Jun. 20, 2003.
Application No. 10/600,290 is a division of application No. 09/447,966, filed on Nov. 23, 1999, now Pat. No. 6,627,616.
Application No. 09/447,966 is a continuation-in-part of application No. 09/391,260, filed on Sep. 7, 1999, now abandoned.
Application No. 09/391,260 is a division of application No. 08/975,573, filed on Nov. 21, 1997, now Pat. No. 6,265,387.
Application No. 08/975,573 is a continuation of application No. 08/571,536, filed on Dec. 13, 1995, now abandoned.
(63) Continuation-in-part of application No. 10/855,175, filed on May 27, 2004.
(60) Provisional application No. 60/473,654, filed on May 28, 2003.
(60) Provisional application No. 60/500,211, filed on Sep. 4, 2003.
(60) Provisional application No. 60/005,091, filed on Oct. 11, 1995.

(51) Int. Cl. A61K 48/00; C12N 15/85

(52) U.S. Cl. 514/44; 435/455

(57) ABSTRACT

Chimeric antitumor compounds for the treatment of cancer are disclosed. The chimeric compounds may be encoded by a nucleic acid. Delivery of the nucleic acid to cells in vivo provides for in vivo production of the antitumor compounds.

### CROSS-REFERENCE TO RELATED APPLICATIONS

This application is a continuation-in-part of application Ser. No. 10/855,175, filed May 27, 2004, and a continuation-in-part of application Ser. No. 10/600,290, filed Jun. 20, 2003, which is a divisional of application Ser. No. 09/447,966, filed Nov. 23, 1999, issued as U.S. Pat. No. 6,627,616, which is a continuation-in-part of application Ser. No. 09/391,260, filed Sep. 7, 1999, abandoned, which is a divisional of application Ser. No. 08/975,573, issued as U.S. Pat. No. 6,265,387, which is a continuation of application Ser. No. 08/571,536, filed Dec. 13, 1995, abandoned. application Ser. No. 10/855,175 claims the benefit of U.S. Provisional Application No. 60/473,654 filed May 28, 2003 and U.S. Provisional Application No. 60/500,211, filed on Sep. 4, 2003, and application Ser. No. 08/571,536 claims the benefit of U.S. Provisional Application No. 60/005,091 filed Oct. 11, 1995.

### BACKGROUND OF THE INVENTION

In the year 2000, approximately 10 million cancers were diagnosed and an estimated 6.2 million cancer-related deaths were reported worldwide. There is an overwhelming need to develop novel therapeutic strategies that bring greater effective disease management and cures.

Solid tumors require the development of a neovascular network in order to progress beyond a minimal size. Primary tumor growth and subsequent metastases therefore require persistent new blood vessel growth. Under typical physiological conditions, angiogenesis is a highly regulated process that is dependent on the balance of pro- and anti-angiogenic modulators. Angiogenesis normally occurs during events such as embryonic development and wound healing. Pathogenic angiogenesis occurs in diseases such as diabetic retinopathy, rheumatoid arthritis, and cancer (Holmgren 1995).

Several proteins are now known to activate endothelial cell growth and movement. These factors are provided endogenously during normal angiogenic events. They include angiogenin, epidermal growth factor, platelet-derived endothelial cell growth factor, estrogen, IL8, prostaglandin E1 and E2, TNFα, vascular endothelial growth factor (VEGF), basic-fibroblast growth factor (bFGF), and granulocyte colony-stimulating factor. In addition, there are many other gene products, ranging from transcription factors to the Notch family members that are essential during new vessel formation. VEGF and bFGF also act as anti-apoptotic factors for the newly formed blood vessels, since they induce expression of anti-apoptotic molecules such as Bcl-2, promoting endothelial cell survival.

The presence of angiogenic factors is not sufficient to initiate new vascular growth. The influence of proangiogenic factors is counterbalanced by a number of inhibitory agents, or antiangiogenic factors. The net result of these opposing factors on the vascular endothelial cell determines the outcome of angiogenesis homeostasis. Several known antiangiogenic factors include endostatin (the cleavage product of collagen), angiostatin (the cleavage product of plasminogen), metalloproteinase inhibitors, interferons (α, β, and γ), calreticulin, certain chemokines (such as IP-10 and Mig), vasostatin, the proapoptotic Bax and interleukin 12 (IL12). Some of these factors inhibit angiogenesis by directly affecting endothelial cell proliferation and survival.

Tumors require and induce a chronic state of angiogenesis in order to establish a neovascular network capable of supporting tumor progression. In the absence of new blood vessel formation, tumor clones are confined to a diameter of about ˜11.5 mm. Gradual tumor expansion causes a progressive central hypoxia as tumor cell proliferation outgrows the capacity of the host vasculature. The hypoxia induces the expression of proangiogenic factors resulting in pathogenic angiogenesis. As tumor neovascularization is an essential process for tumor growth and progression, therapy that is able to prevent or disrupt tumor angiogenesis has important clinical implications. Unfortunately, angiogenesis inhibitors, like angiostatin, endostatin, and others, have been found to be insufficient to induce meaningful antitumor responses.

Tumor cells secrete cytokines and angiogenic molecules that can alter the expression of adhesion molecules and surface markers on endothelium growing in tumors. During tumor angiogenesis, elevated expression of adhesion receptor integrin αvβ3 has been detected in endothelial cells in the tumor. (Brooks 1994). Integrins are important receptors for extracellular matrix (ECM) proteins such as fibronectin, laminins, collagens or vitronectin. Several integrins bind to ECM proteins present in the basal membrane of mature vessels (e.g., laminins or collagens), while other integrins, like αvβ3, bind to ECM proteins present at sites of angiogenesis (e.g., fibronectin or vitronectin). The αvβ3 integrin pair is targeted and bound by the Arg-Gly-Asp (RGD) peptide sequence motif. Some naturally occurring αvβ3 ligands, such as thrombospondin-1 and -2, the MMP-2 proteolytic product PEX, tumstatin, and angiostatin, are known inhibitors of angiogenesis (Ruegg 2004). Pharmacological antagonists of αvβ3 are also known inhibitors of angiogenesis (Brooks 1994, MacDonald 2001). An anti-αvβ3 function-blocking monoclonal antibody (LM609) or an RGD αvβ3/αvβ5-specific antagonistic cyclic peptide (Cilengitide/EMD121974) suppressed tumor angiogenesis in the mouse (Raguse 2004, Dechantsreiter 1999). The ability of the RGD motif to preferentially target and bind tumor neovasculature has prompted utilizing RGD as a targeting mechanism to deliver other moieties, such as chemotherapeutics, to the tumor microenvironment.

Interleukin 12 (IL12) is another protein known to mediate antiangiogenic and immunological antitumor effects (Trinchieri 1995, Trinchieri 2003). In animal models, IL12 treatment has also been shown to stimulate tumor regression and rejection. However, an initial IL12 Phase II clinical study unexpectedly demonstrated lethal toxicities with IL12 protein therapy. Five consecutive daily injections of 1 μg recombinant murine IL12 (rmIL12) protein in mice resulted in nearly 100% mortality. Interestingly, gene therapy approaches using naked DNA to express IL12 have demonstrated antitumor responses against several murine tumors without IL12-associated toxicity. Nonviral intravascular hydrodynamic IL12 gene delivery can induce IL12 serum concentrations of >10 μg/mL without lethal toxicity (Rakhmilevich 1997, Weber 1999, Shi 2002. Weber 2004, Rakhmilevich 1999, Lui 2002).

IL-12 is a disulfide-linked heterodimeric cytokine composed of a light chain (p35) and a heavy chain (p40) produced mainly by antigen-presenting cells (APCs) such as dendritic cells (DC). IL12 stimulates T lymphocytes and NK cells to induce a T helper type 1 (Th1) response. The activated NK and T cells produce IFNγ, which in turn induces production of CXC chemokines: IFNγ-inducible protein 10 (IP-10/CXCL10), monokine-induced-by-IFNγ (MIG/CXCL9), and IFN-inducible T cell α chemoattractant (I-TAC/CXCL 11), as well as additional IL12. In addition to activating NK and T cells, IL12 functions to attract and maintain lymphocytes in areas of sufficient IL12 concentration and enhances production of other pro-inflammatory cytokines such as granulocyte/macrophage colony-stimulating factor (GM-CSF), IL8, and IL18.

To improve the activities of antitumor proteins, such as IL12, and ameliorate their toxicities, it has been proposed to target IL12 to a tumor using RGD peptides (Dickerson et al. US-2003-0077818-A1). The RGD peptide has been utilized as a targeting ligand to increase intratumoral concentration of therapeutic agents like the anticancer drug doxorubicin (Schiffelers 2003). In an effort to target adenovirus gene therapy to the tumor microenvironment, recombinant adenoviruses that incorporate the RGD motif into the HI loop of the fiber knob have been generated (Witlox 2004). Utility of such tumor-specific targeting with RGD-directed therapies is not restricted to tumor neovascular endothelial cells, but also applies to integrin positive tumor cells like melanoma and ovarian cancer (Wu 2004). The feasibility of RGD-directed tumor vascular-targeting of cytokines was recently demonstrated with tumor necrosis factor α (TNFα; Curnis 2004, Zarovni 2004). Intramuscular (i.m.) gene delivery of RGD-TNFα DNA in mice was ineffective at producing detectable systemic levels of TNFα in serum, modest yet significant growth delay of B16 i.d. tumors was noted (Zarovni 2004).

Several natural compounds have been identified that are active suppressors of cancer cell growth, induce anti-angiogenic effects, or induce immune responses directed against tumor cells (e.g., Müillerian inhibitory substance, disintegrins, cytokines). Several problems have prevented successful clinical application of such biologicals. Generally, these compounds are difficult to produce in large quantities. They can also be problematic to administer to patients. For instance, intravenous bolus administration results in undesirable pharmacokinetics and can lead to severe side effects. Often, systemic toxicity prevents dosing at levels required for therapeutic effect. In vivo gene delivery offers another approach for cancer treatment, by enabling the patients' own cells to produce antitumor proteins.

Gene therapy is the purposeful delivery of genetic material to cells for the purpose of treating disease or for biomedical investigation and research. Gene therapy includes the delivery of a polynucleotide to a cell to express an exogenous nucleotide sequence, to inhibit, eliminate, augment, or alter expression of an endogenous nucleotide sequence, or to produce a specific physiological characteristic not naturally associated with the cell. A number of techniques have been explored for delivery of DNA encoding therapeutic genes to cells in mammals. These techniques include direct injection of naked DNA into tissue (Wolff et al. 1990), especially muscle, the “gene gun”, electroporation, the use of viral vectors, and cationic liposome and polymers. These techniques however, suffer from delivery to too few cells and/or toxicity. While highly effective in vitro, cationic DNA-containing complexes generally have been of limited success in vivo because their large size and positive charge have an adverse influence on biodistribution. Delivery of genetic material to cells in vivo is also beneficial in basic research into gene function as well as for drug development and target validation for traditional small molecule drugs.

### SUMMARY OF THE INVENTION

In a preferred embodiment, we describe a process for the treatment of cancer comprising: forming a polynucleotide encoding a chimeric protein comprising a targeting moiety and an antitumor moiety and delivering the polynucleotide to extravascular cells in a mammal by injecting the polynucleotide into a vessel in a mammal. The chimeric protein comprises a targeting subunit and an effector subunit. The effector subunit comprises a peptide, protein or protein subunit known to intrinsically possess or elicit antitumor activity. The targeting moiety comprises a peptide, protein, or protein subunit known to target tumor cells, tumor associated cells, cells known to aid tumor cell growth or cells with antitumor activity. The polynucleotide is rapidly injected into an efferent or afferent vessel of the target tissue in a large volume. The volume of the injected solution and rate of the injection result in increased permeability of the target tissue vasculature thereby increasing extravascular fluid in the target tissue and the movement of the polynucleotide out of the vessel and into extravascular parenchymal cells. Delivery may be enhanced by inhibiting fluid flow from the target tissue during the injection.

In a preferred embodiment, we describe an in vivo process for the delivery of polynucleotides encoding antitumor proteins to parenchymal cells in a mammal comprising: injecting the molecules or complexes in a solution into a vessel, wherein the volume and rate of the injection results in increasing permeability of the vessel thus providing for delivery of the polynucleotides to cells outside the vessel. Increasing vessel permeability and increasing the volume of extravascular fluid in the target tissue may further comprise inhibiting the flow of fluid out of a target tissue during injection of the polynucleotide.

In a preferred embodiment, the polynucleotide is expressed in vivo and the gene product is secreted into the circulation. The targeting moiety of the expressed fusion protein provides increased concentration of the effector moiety in a desired tissue region or phenotypic cell population, thereby reducing systemic concentration, i.e. an increase in the therapeutic index. The desired region can be a tumor or tumor microenvironment, cells or tissue associated with a tumor, cells necessary for tumor growth, or cells capable of antitumor activity. Lower systemic concentration reduces toxicity of the effector subunit. A preferred fusion protein transgene encodes an RGD targeting subunit and an IL12 effector subunit.

In a preferred embodiment, the targeting moiety possesses antitumor activity in addition to its targeting function. A combination of two antitumor activities into a single chimeric protein can result in additive and synergistic antitumor activity.

In a preferred embodiment, we describe a method for treatment of cancer comprising providing for in vivo generation of chimeric antitumor proteins. The chimeric protein comprises a subunit known to have antitumor properties and a subunit known to target tumor cells, cells associated with the tumor, cells necessary for tumor growth, or cells known to have antitumor activity. In vivo generation is provided by delivering to a mammal a polynucleotide comprising an expression cassette encoding the chimeric antitumor protein. The vector can be a naked polynucleotide or a polynucleotide associated with a non-viral vector. The polynucleotide vector is delivered by injecting the polynucleotide into a vessel of the mammal. The polynucleotide is injected into an efferent or afferent vessel of a target tissue in a volume and rate that result in increased permeability of the target tissue vasculature and increased extravascular fluid in the target tissue. Delivery may be enhanced by inhibiting fluid flow out of the target tissue during the injection. The cells to which the polynucleotides are delivery need not be the cells targeting by the fusion protein targeting subunit. The fusion protein transgene may be expressed in a cell and the encoded protein secreted into the circulation.

In a preferred embodiment, we describe hybrid molecules for the treatment of ovarian cancer comprising: chimeric antitumor molecules that are capable of targeting ovarian tumors. These chimeric molecules contain peptide, protein subunit, protein fragment, or full-length protein targeting subunits or other targeting moieties that bind to ovarian tumors through interaction with cell surface molecules. Specific examples include: Anti-Müllerian hormone (AMH, also known as Müllerian inhibitory substance), and moieties known to bind the AMH-receptor, epidermal growth factor receptors HER-1 or HER-2, transmembrane Notch ligand Jagged2, cell adhesion molecule L1CAM, the heat-shock protein 90 kDa HSP90, epithelial cell adhesion molecule EpCAM, CA-125 molecule, mucins MUC1 or MUC16, folate binding protein (FBP), carcinoembryonic antigen (CEA), Tag-72 molecule, Lewis-Y antigen, and cancer-testis antigens NY-ESO-1 or LAGE-1. The moieties that bind ovarian tumor cells are linked to antitumor compounds. The chimeric molecules provide targeting of the biological antitumor compound to the ovarian cancer cells via the targeting moiety, thus increasing the therapeutic index significantly and decreasing toxicity. In a preferred embodiment, the antitumor compound is a peptide, protein subunit, protein fragment or full-length protein. In a preferred embodiment, hybrid anti-ovarian cancer molecule is a Anti-Müllerian Hormone-IL12 fusion protein (AMH-IL12). In a preferred embodiment, the chimeric molecule consists of a protein encoded by a nucleic acid sequence. In a preferred embodiment, a nucleic acid sequence encoding the anti-ovarian cancer chimeric molecule is delivered to a cell in vivo wherein the hybrid molecule is expressed.

Further objects, features, and advantages of the invention will be apparent from the following detailed description when taken in conjunction with the accompanying drawings.

### BRIEF DESCRIPTION OF THE FIGURES

FIG. 1A-1D. Schematic diagram of catheter-mediated intravenous injection of nucleic acids into mammalian limb A) IV delivery to distal hind limb of rats. B) IV delivery to distal hind limb of dog. C) IV delivery to distal hind limb of primate. D) IV delivery to distal hind limb of human. Left panel in each illustrates major veins of the limb. Occlusion sites and injection sites shown in the diagrams are for illustrative purposes. Different occlusion and injection sites are possible as indicated in the description and examples.

FIG. 2 Illustration of the expression cassette for the pRGD-IL12(p40) plasmid (i.e., pMIR305). CMVp/intron=cytomegalovirus immediate early promoter/truncated intron A; p35=murine IL12-p35 subunit cDNA; IRES=Internal Ribosomal Entry Site; p40=murine IL12-p40 subunit cDNA; p(A)=rabbit beta-globin polyadenylation signal sequence.

FIG. 3 Flow cytometry histogram illustrating RGD-IL12 binding to αvβ3 integrins. (A) αvβ3 expressing M21 human melanoma cells incubated with anti-CD51/61-PE (dark grey histogram) or isotype control-PE antibody(open histogram). (B) M21 cells incubated with serum from pCMV-luciferase (open histogram), pNGVCmIL12(light grey), pRGD-IL12(p40) (medium grey), or pRGD-IL12(p35) (dark grey) transfected ICR mice and detected with anti-murine p40/p70 IL12-PE. Numbers indicate specific MFI values.

FIG. 4. Confocal Micrograph showing intratumoral IL12 detection. Panels represent NXS2 tumor sections from separate tumor-bearing mice that received (A) Ringer's solution, (B) plasmid pNGVCmIL12, and (C) plasmid pRGD-IL12(p35). Upper frames are images of FITC (IL12) staining. Lower frames show composite images for nuclear, actin, and murine IL12 staining. X630 magnification.

FIG. 5. Graph illustrating tumor progression in mice treated with A) pRGD-mIL12p40 transgene, B) pRGE-mIL12p40 transgene, C) pRGD-chIL12 transgene, and D) pRGE-chIL12 transgene.

### DETAILED DESCRIPTION

We have developed an intravascular process for the delivery of polynucleotides encoding antitumor chimeric protein to extravascular cells of a mammal in vivo. The invention relates to the use of the vascular system to delivery the polynucleotides to a broader distribution of extravascular cells than is possible with direct injection techniques. The polynucleotide is injected in a solution into an afferent or efferent vessel of a target tissue. The volume of the injection solution and the rate of injection are selected to increase permeability of the target tissue vasculature and increase the volume of extravascular fluid in the target tissue. Delivery is enhanced by impeding fluid flow out of the target tissue during the injection. Using this process, we show delivery of naked polynucleotides and non-viral complexes to parenchymal cells outside a vessel following injection into the lumen of the vessel (U.S. application Ser. No. 10/600,290, incorporated herein by reference).

Using the described processes, extravasation of polynucleotide out of vessels and delivery to cells of the surrounding parenchyma is increased. The method can be used to delivery polynucleotides in vivo to parenchymal cells of tissues selected from the list comprising: liver, kidney, heart, lung, skeletal muscle, prostrate, spleen, and diaphragm.

Delivery of genes encoding chimeric antitumor proteins by these methods can be used in the treatment of tumors. The chimeric proteins comprise a targeting moiety and an effector moiety. The effector moiety or subunit of the chimeric protein comprises any peptide, protein or protein subunit known to possess, elicit, induce, or mediate antitumor or anticancer activity. The targeting moiety or subunit of the chimeric protein associates with tumor cells, cells in the region of a tumor, cells that are beneficial or necessary for tumor cell growth or maintenance, or antitumor effector cells. By associating with these cells, the targeting moiety localizes the effector moiety to the tumor, tumor microenvironment, or tumor associated cells, thereby concentrating the effector moiety activity to the desired region. Efficacy of anticancer proteins can be enhanced by targeting the agent to the cancer cells or tumor area. Because many antitumor proteins are potentially toxic when delivered systemically, targeting the effector moiety reduces their systemic concentration, thereby reducing toxicity and increasing therapeutic indices of the designated effector subunits of the chimeric proteins.

Using recombinant DNA technology readily available in the art, it is possible to create genes encoding fusion proteins which combine antitumor activity (effector moiety; e.g., IL12, TNF-α, contortrostatin) with a targeting signal (targeting moiety; e.g., anti-CA125 antibody, RGD, AMH). A large number of different targeted antitumor fusion proteins can be generated by combining different targeting and effector moieties. The gene can further be designed such that it is expressible in a mammalian cell and such that the expressed fusion protein is secreted by the cell. Delivery of the gene to a cell in the mammal results in production of the antitumor fusion protein and secretion of the protein into the circulation. The targeting moiety then enhances localization the effector moiety subunit to the desired location. Because the gene encodes a fusion protein that may be released into the blood or lymphatic vasculature, it is not required that the gene itself be delivered to the tumor or tumor associated cell.

Delivery of an antitumor chimeric proteins by gene therapy offers several advantages over other tumor treatment strategies. Plasmid DNA preparation is faster, easier, and cheaper than is typically possible for purified protein therapeutics. Second, possible sustained expression of the protein in the mammal allows for prolonged systemic availability of the therapeutic molecule. This continuous dosing provides for decreased frequency of administration and potentially more uniform dosing with lowered toxicity. In contrast, recombinant protein production and purification can be prohibitively expensive and recombinant protein-based therapeutics are typically cleared from the circulation rapidly.

Vessels comprise internal hollow tubular structures connected to a tissue or organ within the body of an animal, including a mammal. Bodily fluid flows to or from the body part within the lumen of the tubular structure. Examples of bodily fluid include blood, lymphatic fluid, or bile. Vessels comprise: arteries, arterioles, capillaries, venules, sinusoids, veins, lymphatics, and bile ducts. Afferent vessels are directed towards the organ or tissue and in which fluid flows towards the organ or tissue under normal physiological conditions. Conversely, efferent vessels are directed away from the organ or tissue and in which fluid flows away from the organ or tissue under normal physiological conditions. In the liver, the hepatic vein is an efferent blood vessel since it normally carries blood away from the liver into the inferior vena cava. Also in the liver, the portal vein and hepatic arteries are afferent blood vessels in relation to the liver since they normally carry blood towards the liver. A vascular network consists of the directly connecting vessels supplying and/or draining fluid in a target organ or tissue. For injection into an artery, the target tissue is the tissue that the artery normally supplies with blood. For injection into a vein, the target tissue is the tissue from which the vein drains blood. For delivery to the liver, for example, the injection solution can be inserted in an antegrade direction into the hepatic artery or the portal vein, or via retrograde injection into the hepatic vein. For delivery to the liver, the polynucleotide can also be injected into the bile duct. For many tissues, the solution is injected in an antegrade direction into a afferent vessel and in a retrograde direction into a efferent vessel.

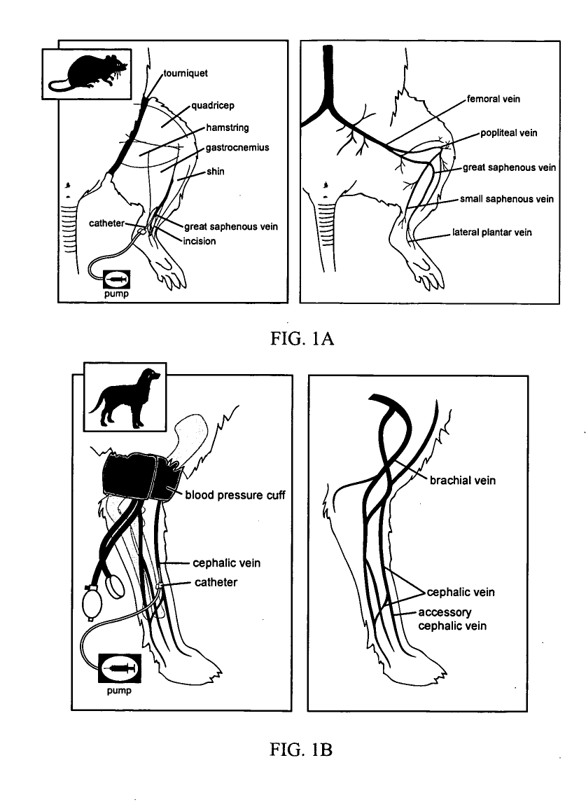

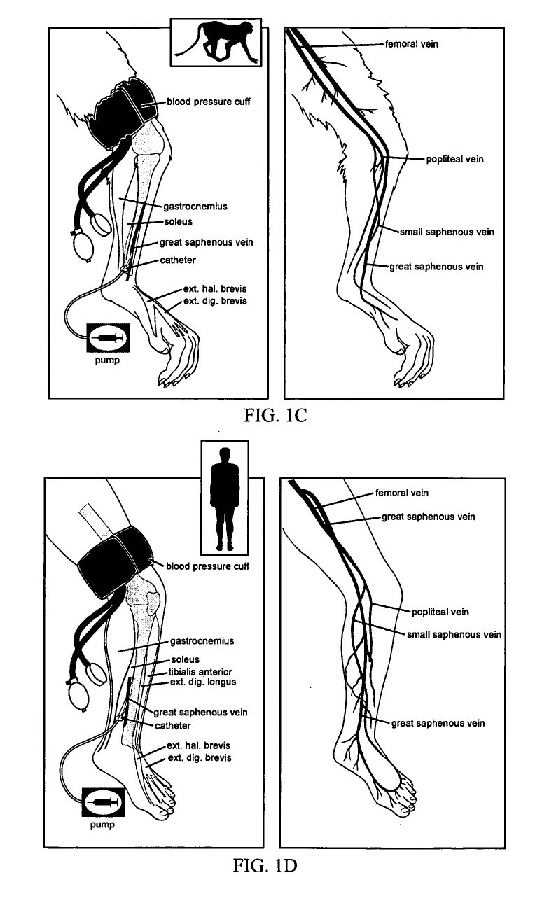

For some tissues, such as limb skeletal muscles, the presence of valves in an efferent vein, make retrograde injection into the vein undesirable. We show that for delivery of polynucleotides to limb extravascular cells, antegrade injection of the solution into either an artery or a vein provides efficient delivery of polynucleotides to extravascular limb cells (U.S. application Ser. No. 10/855,175, incorporated herein by reference). The intravascular limb delivery method comprises: impeding fluid flow out of a target limb, inserting an injection device into a vessel in the limb distal to the occlusion, and injecting a solution containing the polynucleotide into the vessel in an antegrade direction. The polynucleotides are delivered to cells distal to the occlusion. Cells located distal to the occlusion are those cells located between the occlusion and the end of the limb that is farther from the heart. For injection into an artery, the solution is typically injected near the occlusion. For injection into a vein, the solution may be injected as shown in FIG. 1. For injection into a vein of the hand, foot or joint, the solution may be injected in a retrograde direction. Venous injection combined with the use of a cuff for impeding blood flow provides a non-surgical method for polynucleotide delivery. Vessels of the venous system have reduced vessel wall thickness relative to comparable arterial vessels and they can be made more permeable than the arterial system thus allowing increased delivery to extravascular locations with decrease injection volume. For certain clinical indications, where the arterial system displays vascular pathology (arteriosclerosis, atherosclerosis, and single or multiple partial or total occlusions), the venous system represents a more attractive delivery conduit to deliver polynucleotides to limb skeletal muscle cells.

Inserting an appropriate volume of injection solution into a vessel at an appropriate rate, optionally together with occlusion of fluid flow from the target tissue, increases permeability of vasculature in the tissue to the injection solution and the polynucleotides therein and results in delivery of the polynucleotide to extravascular cells. Permeability is the propensity for macromolecules to move out of a vessel and enter the extravascular space. The injection volume and injection rate are dependent upon: the size of the animal, the size of the vein into which the solution is injected, and the size and/or volume of the target tissue. Larger injection volumes and/or higher injection rates are required for larger target sizes. For delivery to larger animals, injection of larger volumes is expected. The volume and injection rate can also be affected by the nature of the target tissue. For example, delivery to liver may require less volume because of the porous nature of the liver vasculature. The precise volume and rate of injection into a particular vessel, for delivery to a particular target tissue of a given mammal species, may be determined empirically. The described methods provide for more even distribution of polynucleotides to cells throughout a target tissue than is possible with direct parenchymal injections.

Because vasculature may not be identical from one individual to another, methods may be employed to predict or control appropriate injection volume and rate. Injection of iodinated contrast dye detected by fluoroscopy can aid in determining vascular bed size. MRI (magnetic resonance imaging) can also be used to determine bed size. If the target tissue is a limb, volume displacement can be used to determine its size. Also, an automatic injection system can be used such that the injection solution is delivered at a preset pressure or rate. For such a system, pressure may be measured in the injection apparatus, in the vessel into which the solution is injected, in a branch vessel within the target tissue, or within a vein or artery within the target tissue.

By increasing the amount of polynucleotide injected and the volume of injection, the methods described for intravascular delivery of polynucleotides to small mammals such as rodents or rhesus monkeys are readily adapted to use in larger animals. Injection rate may also be increased for delivery to larger mammals. Conversely, for delivery to smaller animals, the injection volume and/or rate is reduced. Intravascular delivery of polynucleotides to extravascular cells is increased by impeding the outflow of fluid from the tissue during injection of the polynucleotide. For example, the solution may be injected into an afferent vessel supplying a target tissue while efferent vessels of the tissue are occluded. Conversely, the polynucleotides may be injected into an efferent vessels of a target tissue with occlusion of corresponding afferent vessel(s) of the target tissue. The occlusion may be released immediately after injection, within 2 minutes of injection, within 5 minutes of injection, within 10 minutes of injection, or may be released only after a determined length of time which does not result in tissue damage due to ischemia.

In the heart, efficient delivery through a coronary vein does not require occluding free blood flow through the corresponding artery. In this case, the microcapillary bed generates sufficient resistance to increase vessel permeability following solution injection. Similarly, it is possible to delivery polynucleotides to rodent liver cells via injection of polynucleotides into tail vein without occluding afferent and efferent liver vessels. For delivery to extravascular limb cells, injection of the solution without impeding outflow results in much decreased transfection of the target cells.

Occlusion of fluid flow, by balloon catheters, clamps, or non-invasive cuffs can limit or define the target tissue. One example of a non-invasive cuff is a sphygmomanometer, which is normally used to measure blood pressure. Another example is a tourniquet. A third example is a modified sphygmomanometer cuff containing two air bladders such as is used for intravenous regional anesthesia (i.e. Bier Block). Double tourniquet, double cuff tourniquet, oscillotonometer, oscillometer, and haemotonometer are also examples of cuffs. A sphygmamanometer can be inflated to a pressure above the systolic blood pressure, above 500 mm Hg or above 700 mm Hg or greater than the intravascular pressure generated by the injection. Non-invasive cuffs allow the occlusion of fluid flow from a limb without the invasive placement of clamps on limb vessels.

A syringe needle, cannula, catheter or other injection device may be used to inject the polynucleotide into the vessel. Single and multi-port injectors may be used, as well as single or multi-balloon catheters and single and multilumen injection devices. A catheter can be inserted at a distant site and threaded through the lumen of a vessel so that it resides in or near a target tissue. The injection can also be performed using a needle that traverses the skin and enters the lumen of a vessel.

The polynucleotide is injected in a pharmaceutically acceptable solution. Pharmaceutically acceptable refers to those properties and/or substances which are acceptable to the mammal from a pharmacological/toxicological point of view. The phrase pharmaceutically acceptable refers to molecular entities, compositions and properties that are physiologically tolerable and do not typically produce an allergic or other untoward or toxic reaction when administered to a mammal. Preferably, as used herein, the term pharmaceutically acceptable means approved by a regulatory agency of the Federal or a state government or listed in the U.S. Pharmacopeia or other generally recognized pharmacopeia for use in animals, and more particularly in humans.

The injection solution may further contain a compound or compounds which may aid in delivery and may or may not associate with the polynucleotide. The polynucleotides may be naked polynucleotides or they may be in association with components that aid in delivery, such as non-viral transfection agents.

The composition of the injection solution can depend on the nature of the molecule or complex that is to be delivered. We have observed that certain complexes, especially complexes formed by electrostatic interaction between the complex components, may be delivered more efficiently using low salt injection solutions.

Other agents known in the art may be used to further increase vessel permeability, including drugs or chemicals and hypertonic solutions. Drugs or chemicals can increase the permeability of the vessel by causing a change in function, activity, or shape of cells within the vessel wall; typically interacting with a specific receptor, enzyme or protein of the vascular cell. Other agents can increase permeability by changing the extracellular connective material. Examples of drugs or chemicals that may be used to increase vessel permeability include, but are not limited to, histamine, vascular permeability factor or vascular endothelial growth factor (VEGF), calcium channel blockers, beta-blockers, and papaverine. The permeability enhancing drug or chemical may be present in the polynucleotide-containing injection solution. An efflux enhancer solution, a solution containing a permeability enhancing drug or chemical, may also be injected into the vessel prior to injection of the solution containing the polynucleotide. Hypertonic solutions have increased osmolarity compared to the osmolarity of blood thus increasing osmotic pressure and causing cells to shrink. Typically, hypertonic solutions containing salts such as NaCl or sugars or polyols such as mannitol are used. Delivery might also be enhanced by pharmacologic agents that cause vasoconstriction or vasodilation. Agents that block or prevent blood clotting (or digest blood clots) may also be injected into the vessel. Enzymes such as collagenases, hyaluronidases, and heparinases may also be used to improve delivery.

The described process may also be used repetitively in a single mammal. Multiple injections may be used to provide delivery to additional tissues, to increase delivery to a single tissue, or where multiple treatments are indicated. Multiple injections may be performed in different sites of the same mammal, within the same site, within the same vessel of the mammal, or within different vessels in the mammal. Sites of vessel occlusion may also be the same or different for multiple injections in the same mammal.

It is particularly noteworthy that the level of transgene expression that can be achieved using the described procedures does not diminish as the procedure is scaled up to larger mammals. In contrast, direct intramuscular injections of plasmid DNA results in high expression levels per gram of muscle in rodents but very low expression levels per gram of muscle in primates (Jiao et al. 1992). Because the method is readily adapted to use in rats, dogs, and nonhuman primates, it is expected that the method is also readily adapted to use in other mammals, including humans.

The injection volume is dependent on the size of the animal to be injected and can be from as little as 1 ml or less for small animals to more than 100 ml for injection into rhesus monkey limb. Injection into larger mammals can require larger injection volume. By way of example, injection of about 1 to about 3 ml into the tail vein of a mouse or about 6 to about 35 ml into the tail vein of rat may be used for delivery of polynucleotides to the liver. For delivery to mouse hind limb (20-30 g animal total weight), about 0.2 to about 3 ml injection solution at a rate of 0.5-25 ml/min into the saphenous vein results in delivery of polynucleotides to multiple muscle cells throughout the limb. For delivery to rat hind limb, about 8 to about 12 ml injection solution at a rate of 20-75 ml/min into the iliac artery results in delivery of polynucleotides to multiple muscle cells throughout the limb. Also, for delivery to rat hind limb, about 4.5 ml to about 10.5 ml injection solution at a rate of about 3 to about 20 ml/min into the saphenous vein results in delivery of polynucleotides to multiple muscle cells throughout the limb. Injection of about 35 to about 90 ml into a beagle dog (˜9.5 kg total animal weight) limb vein (about 0.8 to about 1.6 ml per gram target tissue) at a rate of about 1.5 ml/sec or higher may be used for delivery to limb skeletal muscle cells. Injection of about 50 to about 500 ml into a beagle dog limb artery (about 3 to about 7 ml per gram target tissue) at a rate of about 100 ml/min or higher may be used for delivery to limb skeletal muscle cells. Injection of about 30 to about 130 ml into a rhesus monkey limb vein (about 0.3 to about 2.0 ml per gram target tissue) at a rate of about 1.5 ml/sec or higher may be used for delivery to limb skeletal muscle cells. Injection of about 70 to about 200 ml or more into a rhesus monkey limb artery (about 1.4 to about 2.3 ml per gram target tissue) may be used for delivery to limb skeletal muscle cells. Injection of about 20 to about 30 ml or more into a pig (30-40 kg animal weight) coronary artery or vein at a rate of about 1 ml/sec or higher may be used for delivery to heart cardiac muscle cells. The volume of solution is limited by the amount that is well tolerated by the mammal and should be chosen such that unacceptable harm is not caused to the tissue or mammal.

The solution containing the polynucleotide is also injected rapidly. Injection times can be as rapid as a few seconds, such as for injection into tail vein of a mouse, to about two minutes for injection into a vessel in the limb of a dog or primate. The injection rate can be from about 0.5 ml/min to about 120 ml/min or higher. The rate of injection is partially dependent the volume to be injected, the size of the vessel into which the solution is to be injected into, and the size of the animal. As with the injection volume, the injection rate should not be greater than that which is well tolerated by the mammal.

Effector moiety: Full-length protein, protein subunit, protein fragment, or peptide that possess functional biological activity in the sense of serving as a broadly defined biological response modifier (BRM) having antitumor activity. BRMs may act directly on tumor cells to induce antitumor activity through processes such as induction of apoptotic or necrotic cell death. Additionally, BRMs may act on non-tumor cell populations that serve as antitumor effector cells, such as lymphocytes, to disrupt tumor growth progression involving direct effector-tumor cell contact or via release of soluble mediators that orchestrate additional and subsequent downstream events that manifest as an antitumor response or activity.

Antitumor activity: comprises any biological activity which reduces, impedes, or disrupts cancer or tumor cell growth. Antitumor activities include, but are not limited to, inhibiting disease progression wherein the disease is cancer, promoting tumor regression and resolution, inhibiting metastatic disease, promoting antiangiogenic activity, and inducing or enhancing immune activity, including enhancing lymphocyte recruitment to a tumor. This activity may be include, but is not limited to, a blockade of tumor cell-cycle progression inhibiting tumor cell replication, reduced or loss in blood flow to tumor microenvironment by antiangiogenic mechanisms that result in severe tumor hypoxia, or direct tumor cell death induced by apoptotic or necrotic pathways.

Exemplary compounds possessing potential antitumor activity include, but are not limited to: interleukins (including IL12), Müllerian inhibitory substance, contortrostatin, growth factors, growth analogs, anti-angiogenic factors, cytokines, immunocytokines, Interferons, Chemokines, Protein/peptide chemotherapeutics, antibodies, hypoxia inducing factors, apoptosis inducers, steroids, glucocorticoids, pro-inflammatory factors, antivirals or antimicrobials, kinase inhibitors, cell-cycle inhibitors, HSP90 inhibitors, histone deacetylase inhibitors, chemoattractants, small immunoproteins. (oncolytic substances, angiogenic compounds)

Targeting moiety: Targeting moieties comprise peptide sequence motifs, proteins, or protein subunits or compounds known to have affinity to receptors or other cell surface molecules present on the target cell. A target cell may be a tumor cell, non-tumor cell within or adjacent to the tumor microenvironment, cell involved in development of tumor neovasculature, tumor infiltrating lymphocyte, or effector cell capable of mediating an antitumor effect toward the tumor cell. Examples of targeting moieties include: Anti-Müllerian hormone, RGD-containing peptides, malarial peptide, etc. The targeting moiety can also contain antitumor activity.

Cell-surface receptors and molecules on target cells are utilized for localizing the fusion protein to the target cell through affinity binding interactions with the targeting moiety of the fusion protein. Specific receptors and molecules that are targeted include those characterized and unidentified present on tumor cell, non-tumor cells within or adjacent to the tumor microenvironment, cells involved in development of tumor neovasculature, tumor infiltrating lymphocytes, or effectors cells capable of mediating an antitumor effect toward the tumor cell. Cell-surface receptors which are present predominantly on the target cell or are expressed at elevated levels on the target cell are preferred. Potential target cell receptors/molecules include, and are not limited to: integrins, surface-expressed tumor associated antigen (TAA), HER-2/neu, GD2 disialoganglioside, CEA, EpCAM, CD20, CD71 (transferrin receptor), MHC class I/TAA peptide complex, Gp100, CD13, VEGF receptor, Fc receptor, CD25 (IL2 receptor), and Toll-like receptors (TLR). Exemplary targeting moieties include, but are not limited to: integrin binding ligands, RGD peptide, NGR peptide, disintegrin, tail fiber protein. Antibodies or antibody fragments with affinity to the desired receptor may also function as targeting moieties.

In a preferred embodiment, the described processes may by used to treat any tumor, and especially any vascularized tumor. The described delivery processes may also be used to deliver genes encoding proteins possessing antitumor activity but which are not fusion proteins. The described processes for treatment of tumors may further be combined with other antitumor treatment modalities: such as antibody-IL2 immunocytokine therapy, immunotherapy, chemotherapy; and radiotherapy.

By way of example, to illustrate the invention, an exemplary therapeutic fusion protein transgene is described. The RDG-IL12 fusion protein contains an RGD-containing peptide targeting moiety and an IL12 protein effector moiety that stimulates antiangiogenic and immunologic action. The RGD targeting moiety functions as a ligand for the αvβ3 integrin receptor located on neovascular endothelial cells in the region of the tumor. RGD-peptides that are constrained in a preferred cyclic conformation, such as RGD-4C (CDCRGDCFC; SEQ ID 1), show an increased affinity for integrin binding (Dechantsreiter 1999). Mobilizing IL12 to the tumor site by RGD neovascular targeting facilitates therapeutic intratumoral levels of IL12 while lowering systemic exposure and alleviating potential toxicity concerns present with native IL12 treatment. Providing antitumor responsiveness at lowered systemic IL12 concentration should translate into a more favorable therapeutic index with RGD-targeted IL12. Tumor-bearing mammals may also have decreased IL12 sensitivity because of sequestration of the RGD-targeted IL12 to the tumor region.

RGD peptides can directly affect tumor vasculature by inducing endothelial cell death and inhibiting further tumor angiogenesis by disruption of existing tumor neovasculature. Therefore, the RGD-IL12 protein further provides an example of the use of a targeting moiety that also possesses effector moiety/antitumor function.

The RGD-IL12 transgene combines the antiangiogenic and tumor-vasculature-targeting activities of the RGD peptide with the pleiotropic antitumor potential of IL 12 into a single multifunctional fusion molecule expressed in vivo following gene transfer. The RGD-IL12 chimeric protein combines mechanistically distinct antitumor strategies into a single reagent. Because the mechanisms of antitumor action of RGD-peptide and IL12 are independent, multifunctional RGD-IL12 fusion protein may promote an additive or synergistic antitumor benefit. Additional antitumor benefit is achieved by augmented antiangiogenic activity and immunological activation occurring through the increased intratumoral levels of IL12. The RGD-IL12 fusion protein therefore provides improved therapeutic benefit over delivery of the two molecules separately and will lower systemic IL12 levels and associated side-effects.

An exemplary therapeutic anti-ovarian cancer hybrid molecule is an Anti-Müllerian Hormone-biological response modifier (AMH-BRM) fusion protein. More specifically, a AMH-IL12 fusion molecule. Ovarian cancer is the second most common pelvic tumor and the leading cause of death from a gynecologic malignancy. Because of the lack of symptoms in the early stages, two thirds of the patients present with advanced late-stage disease. Despite advances in surgical oncology, chemotherapy, and molecular biology, overall 5-year survival rates are still poor (approximately 30%). Ovarian cancer spreads into the abdomen early in the disease by exfoliation of cancer cells following the natural circulation of peritoneal fluid. Following attachment to the peritoneal surface, the cells grow as surface nodules (peritoneal disseminated ovarian cancer). Cytoreductive, or debulking surgery, is a pivotal component of salvage therapy. Yet, the minimal size of the metastatic tumors that can be subjected to surgery is limited. Whole-abdominal radiation therapy is also considered non-effective with severe toxicity reported. Thus, there is a great need for alternative treatment approaches.

Several natural compounds have been identified that are active suppressors of ovarian cancer cell growth, induce anti-angiogenic effects, or induce immune responses directed against the tumor cells (e.g., Anti-Müllerian Hormone (AMH, also known as Müllerian inhibitory substance, disintegrins, and cytokines). Several problems have prevented successful clinical application of such biologicals. Generally, these compounds are difficult to produce in large quantities and are problematic to administer to patients. For instance, intravenous bolus administration results in undesirable pharmacokinetics and can lead to severe side effects.

Often, systemic toxicity prevents dosing at levels required for therapeutic effect. It is our hypothesis that a gene therapy approach can overcome these limitations and that treatment efficacy can be significantly enhanced by targeting the biological to the ovarian cancer microenvironment.

Creation of fusion proteins that combine an effector moiety with a targeting moiety (e.g., AMH, anti-CA125, or RGD) provide for increased effectiveness of the therapeutic molecule. The Müllerian duct, which forms from coelomic epithelium, develops into the fallopian tubes, uterus, cervix, proximal vagina, and surface epithelium of the ovary in the female. These structures regress in the male embryo as a result of exposure to AMH, which signals through a two-receptor system. The AMH type II receptor (AMHR) is a transmembrane serine-threonine kinase, and controls AMH-binding specificity. AMH-bound AMHR-II phosphorylates the type I receptor, which is responsible for signal transduction and initiates Müllerian duct regression. AMH can inhibit the growth of numerous cancers including: ovarian, cervical, endometrial, and prostate, with all cells expressing the AMHR showing susceptibility to AMH effects. A survey of ascites material indicated that ˜60% of those ovarian cancer patients evaluated had tumor cells able to bind AMH. Murine xenogenic models have shown inhibition of human ovarian cancer tumor progression following in vivo AMH delivery.

The carboxy terminal subunit of AMH is able to exert biological antitumor activity. An AMH-IL12 fusion protein, would possess the complete AMH or the biologically active carboxy terminal subunit as the targeting moiety and an IL12 effector moiety. As with the RGD-IL12 chimeric protein, the targeting moiety, AMH, additionally possesses independent antitumor activity. The AMH-BRM fusion molecule could be produced in vitro and delivered to patients. Alternatively, the an AMH-BRM transgene could be delivered to the patient.

Parenchymal Cells: Parenchymal cells are the distinguishing cells of a gland, organ or tissue contained in and supported by the connective tissue framework. The parenchymal cells typically perform a function that is unique to the particular organ. The term “parenchymal” often excludes cells that are common to many organs and tissues such as fibroblasts and endothelial cells within the blood vessels.

In a liver organ, the parenchymal cells include hepatocytes, Kupffer cells and the epithelial cells that line the biliary tract and bile ductules. The major constituent of the liver parenchyma are polyhedral hepatocytes (also known as hepatic cells) that present at least one side to a hepatic sinusoid and an apposed side to a bile canaliculus. Cells in the liver that are not parenchymal cells include the endothelial cells or fibroblast cells within the blood vessels.

In striated muscle, the parenchymal cells include myoblasts, satellite cells, myotubules, and myofibers. In cardiac muscle, the parenchymal cells include the myocardium (also known as cardiac muscle fibers or cardiac muscle cells) and the cells of the impulse connecting system such as those that constitute the sinoatrial node, atrioventricular node, and atrioventricular bundle.

In a pancreas, the parenchymal cells include cells within the acini such as zymogenic cells, centroacinar cells, basal or basket cells and cells within the islets of Langerhans such as alpha and beta cells.

In spleen, thymus, lymph nodes and bone marrow, the parenchymal cells include reticular cells and blood cells (or precursors to blood cells) such as lymphocytes, monocytes, plasma cells and macrophages.

In the nervous system which includes the central nervous system (the brain and spinal cord) peripheral nerves, and ganglia, the parenchymal cells include neurons, glial cells, microglial cells, oligodendrocytes, Schwann cells, and epithelial cells of the choroid plexus.

In a kidney, parenchymal cells include cells of collecting tubules and the proximal and distal tubular cells.

In the prostate, the parenchyma includes epithelial cells.

In glandular tissues and organs, the parenchymal cells include cells that produce hormones. In the parathyroid glands, the parenchymal cells include the principal cells (chief cells) and oxyphilic cells. In a thyroid gland, the parenchymal cells include follicular epithelial cells and parafollicular cells. In adrenal glands, the parenchymal cells include the epithelial cells within the adrenal cortex and the polyhedral cells within the adrenal medulla.

In the gastrointestinal tract, including the esophagus, stomach, and intestines, the parenchymal cells include epithelial cells, glandular cells, basal, and goblet cells.

In a lung, the parenchymal cells include the epithelial cells, mucus cells, goblet cells, and alveolar cells.

In fat tissue, the parenchymal cells include adipose cells or adipocytes.

In skin, the parenchymal cells include the epithelial cells of the epidermis, melanocytes, cells of the sweat glands, and cells of the hair root.

In cartilage, the parenchyma includes chondrocytes. In bone, the parenchyma includes osteoblasts, osteocytes, and osteoclasts.

Polynucleotide: The term polynucleotide, or nucleic acid or polynucleic acid, is a term of art that refers to a polymer containing at least two nucleotides. Nucleotides are the monomeric units of polynucleotide polymers. Polynucleotides with less than 120 monomeric units are often called oligonucleotides. Natural nucleic acids have a deoxyribose- or ribose-phosphate backbone. An artificial or synthetic polynucleotide is any polynucleotide that is polymerized in vitro or in a cell free system and contains the same or similar bases but may contain a backbone of a type other than the natural ribose-phosphate backbone. These backbones include: PNAs (peptide nucleic acids), phosphorothioates, phosphorodiamidates, morpholinos, and other variants of the phosphate backbone of native nucleic acids. Bases include purines and pyrimidines, which further include the natural compounds adenine, thymine, guanine, cytosine, uracil, inosine, and natural analogs. Synthetic derivatives of purines and pyrimidines include, but are not limited to, modifications which place new reactive groups such as, but not limited to, amines, alcohols, thiols, carboxylates, and alkylhalides. The term base encompasses any of the known base analogs of DNA and RNA. The term polynucleotide includes deoxyribonucleic acid (DNA) and ribonucleic acid (RNA) and combinations of DNA, RNA and other natural and synthetic nucleotides.

Expression cassette: The term expression cassette refers to a natural or recombinantly produced nucleic acid molecule that is capable of expressing a gene or genetic sequence in a cell. An expression cassette typically includes a promoter (allowing transcription initiation), and a sequence encoding one or more proteins or RNAs. Optionally, the expression cassette may include transcriptional enhancers, non-coding sequences, splicing signals, transcription termination signals, and polyadenylation signals. An RNA expression cassette typically includes a translation initiation codon (allowing translation initiation), and a sequence encoding one or more proteins. Optionally, the expression cassette may include translation termination signals, a polyadenosine sequence, internal ribosome entry sites (IRES), and non-coding sequences. Optionally, the expression cassette may include a gene or partial gene sequence that is not translated into a protein. The nucleic acid can effect a change in the DNA or RNA sequence of the target cell. This can be achieved by hybridization, multi-strand nucleic acid formation, homologous recombination, gene conversion, RNA interference or other yet to be described mechanisms.

The term gene generally refers to a nucleic acid sequence that comprises coding sequences necessary for the production of a nucleic acid (e.g., siRNA) or a polypeptide or precursor. A polypeptide can be encoded by a full length coding sequence or by any portion of the coding sequence so long as the desired activity or functional properties (e.g., enzymatic activity, ligand binding, signal transduction) of the full-length polypeptide or fragment are retained. The term also encompasses the coding region of a gene and the including sequences located adjacent to the coding region on both the 5′ and 3′ ends for a distance of about 1 kb or more on either end such that the gene corresponds to the length of the full-length mRNA. The sequences that are located 5′ of the coding region and which are present on the mRNA are referred to as 5′ untranslated sequences. The sequences that are located 3′ or downstream of the coding region and which are present on the mRNA are referred to as 3′ untranslated sequences. The term gene encompasses synthetic, recombinant, cDNA and genomic forms of a gene. A genomic form or clone of a gene contains the coding region interrupted with non-coding sequences termed introns, intervening regions or intervening sequences. Introns are segments of a gene which are transcribed into nuclear RNA. Introns may contain regulatory elements such as enhancers. Introns are removed or spliced out from the nuclear or primary transcript; introns therefore are absent in the mature RNA transcript. Components of a gene also include, but are not limited to, promoters, enhancers, transcription factor binding sites, polyadenylation signals, internal ribosome entry sites, silencers, insulating sequences, matrix attachment regions. Non-coding sequences influence the level or rate of transcription and/or translation of the gene. Covalent modification of a gene may influence the rate of transcription (e.g., methylation of genomic DNA), the stability of mRNA (e.g., length of the 3′ polyadenosine tail), rate of translation (e.g., 5′ cap), nucleic acid repair, nuclear transport, and immunogenicity. Gene expression can be regulated at many stages in the process. Up-regulation or activation refers to regulation that increases the production of gene expression products (i.e., RNA or protein), while down-regulation or repression refers to regulation that decrease production. Molecules (e.g., transcription factors) that are involved in up-regulation or down-regulation are often called activators and repressors, respectively.

Transfection Agent: A transfection agent, or transfection reagent or delivery vehicle, is a compound or compounds that bind(s) to or complex(es) with oligonucleotides and polynucleotides, and enhances their entry into cells. Examples of transfection reagents include, but are not limited to, cationic liposomes and lipids, polyamines, calcium phosphate precipitates, histone proteins, polyethylenimine, polylysine, and polyampholyte complexes. It has been shown that cationic proteins like histones and protamines, or synthetic polymers like polylysine, polyarginine, polyornithine, DEAE dextran, polybrene, and polyethylenimine may be effective intracellular in vitro delivery agents. Typically, the transfection reagent has a component with a net positive charge that binds to the oligonucleotide's or polynucleotide's negative charge. For delivery in vivo, complexes made with sub-neutralizing amounts of cationic transfection agent may be preferred. Non-viral vectors is include protein and polymer complexes (polyplexes), lipids and liposomes (lipoplexes), combinations of polymers and lipids (lipopolyplexes), and multilayered and recharged particles. Transfection agents may also condense nucleic acids. Transfection agents may also be used to associated functional groups with a polynucleotide. Functional groups include cell targeting signals, nuclear localization signals, compounds that enhance release of contents from endosomes or other intracellular vesicles (such as membrane active compounds), and other compounds that alter the behavior or interactions of the compound or complex to which they are attached (interaction modifiers).

The cell targeting signal can be cell receptor ligands, such as proteins, peptides, sugars, steroids and synthetic ligands as well as groups that interact with cell membranes, such as lipids, fatty acids, cholesterol, dansyl compounds, and amphotericin derivatives. The signal may increase binding of a compound to the cell surface and/or its association with an intracellular compartment. Other targeting groups can be used to increase the delivery of the polynucleotide to certain parts of the cell, such as nuclear localization signals.

### EXAMPLES

### Example 1

RGD-IL12pDNAs.

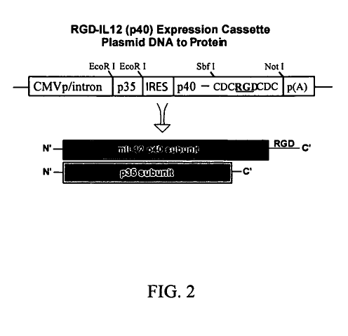

pNGVCmIL12 plasmid (Aldevron, Fargo N.D.) expresses both the p35 and p40 murine IL12 (mIL12) subunits, which combine to form the functionally active IL12 heterodimer. The human cytomegalovirus (CMV) immediate-early promoter drives transcription of both subunits. Between the p35 stop codon and the p40 start codon is the encephalomyocarditis virus Internal Ribosomal Entry Site (IRES), facilitating translation of both subunits from a single transcript (FIG. 2). Two versions of the RGD-mIL12 fusion molecule were made: pRGD-mIL12(p35) gene product has the RGD-4C (CDCRGDCFC, SEQ ID 1) peptide attached to the carboxy-terminus of the p35 subunit; and, pRGD-mIL12(p40) has RGD-4C attached to the carboxy-terminus of the p40 subunit.

controls: To empirically test antitumor efficacy of RGD-mIL12 against RGD plus mIL12 combined gene therapy, we developed additional RGD-mIL12 pDNA constructs to express fusion proteins that have nonfunctional RGD or IL12 components (Table 1). A single amino acid substitution Asp to Glu (RGD-4C to RGE-4C), results in a dramatic reduction in binding affinity of the peptide for the αvβ3 integrin. Expression cassettes which encode a nonfunctional RGE sequence, resulting in pRGE-mIL12p40 and pRGE-mIL12p35, are used as controls.

A chimeric IL12 (chIL12) heterodimer, human p35 IL12 (hp35) subunit with murine p40 IL12 (mp40) subunit has activity on human, but not murine cells. RGD- and RGE-coding plasmids were developed to express chIL12 (murine p40/human p35). The hp35 subunit was obtained from the human IL12-expressing pUMVC3-hIL12 plasmid (Aldevron).

Delivery and expression of RGD-IL12 in vivo. Expression vectors were delivered to mouse liver cells by hydrodymamic tail vein (HTV) delivery (U.S. Application US-2001-0004636; Zhang et al. Gene Therapy 2000; Zhang et al. Gene Therapy 2004; Zhang et al. Hum Gene Ther 1999). For these experiments, 1 ml solution containing the polynucleotide to be delivered per 10 animal body weight was injected into the tail vein of mice in less than 10 sec. Circulating serum levels of mIL12p40 were measured to determine the expression levels for each of the fusion protein expression cassettes. Groups (n=4) of ICR mice received 0.2 or 20 μg of pDNA by HTV delivery and were bled at various time-points. The individual serum samples were pooled for each treatment group at each time-point and quantitated by murine IL12p40 ELISA (R&D Systems, Minneapolis, Minn.). Elevated levels of serum mIL12p40 were detected in the sera of all mice (Table 1). Table 1 shows that the 24 hr serum mIL12p40 levels are diminished as compared to the earlier 12 hr time-point, consistent with reports that maximal gene expression is observed during the first 8-12 hr following HTV gene delivery. Delivery of pNGVCmIL12 consistently resulted in approximately a 3-17 fold higher serum level of mIL12 as compared to pRGD-IL12p40 and pRGD-IL12p35 at both pDNA doses tested.

TABLE 1
Gene Product Characterization

| | HTV | Functional² | | Attach | Serum IL12 level⁴ | | Serum IFNγ⁵ | | αvβ3 |
|---|---|---|---|---|---|---|---|---|---|
| plasmid | dose¹ | RGD | mIL12 | site³ | 12 hr | 24 hr | 48 hr | 96 hr | binding⁶ |
| pRGD- | 20 | + | + | mp40 | 2593 | 302 | 4.4 | 17.5 | 18 |
| mIL12p40 | 0.2 | | | | 19 | 7 | 0.3 | <0.1 | n.d. |
| pRGE- | 20 | − | + | mp40 | 20109 | 11066 | 2.3 | 9.6 | 8 |
| mIL12p40 | 0.2 | | | | 392 | 158 | 2.2 | 1.1 | n.d. |
| pRGD- | 20 | + | − | mp40 | 1839 | 365 | <0.1 | <0.1 | 20 |
| chIL12p40 | 0.2 | | | | 6 | 4 | <0.1 | <0.1 | n.d. |
| pRGE- | 20 | − | − | mp40 | 10907 | 3342 | 0.9 | <0.1 | 8 |
| chIL12p40 | 0.2 | | | | 64 | 13 | <0.1 | 0.5 | n.d. |
| pRGD- | 20 | + | + | mp35 | 3571 | 1112 | 1.3 | 1.5 | 47 |
| mIL12p35 | 0.2 | | | | 46 | 8 | 0.3 | 0.1 | n.d. |
| pRGE- | 20 | − | + | mp35 | 8153 | 1471 | 2.8 | 9.6 | 8 |
| mIL12p35 | 0.2 | | | | 142 | 95 | 3.6 | 0.7 | n.d. |
| pRGD- | 20 | + | − | mp35 | 10127 | 3744 | <0.1 | <0.1 | 25 |
| chIL12p35 | 0.2 | | | | 231 | 68 | <0.1 | <0.1 | n.d. |
| pRGE- | 20 | − | − | mp35 | 22708 | 8651 | 0.5 | <0.1 | 8 |
| chIL12p35 | 0.2 | | | | 236 | 62 | <0.1 | <0.1 | n.d. |
| pNGVC- | 20 | n.a. | − | n.a. | 10788 | 6265 | 2.4 | 18.1 | n.d. |
| mIL12 | 0.2 | | | | 334 | 121 | 1.0 | <0.1 | n.d. |
| pchIL12hp35 | 20 | n.a. | − | n.a. | 14274 | 9276 | 0.1 | <0.1 | n.d. |
| | 0.2 | | | | 323 | 78 | <0.1 | <0.1 | n.d. |

¹dose in μg pDNA injected
²indicates presence or absence of functional RGD peptide or mouse IL12 activity
³indicates IL12 subunit to which RGD is attached
⁴values represent serum mIL12 levels in ng/ml as determined by mIL12p40 ELISA
⁵values represent IFNγ levels in ng/ml as determine by ELISA (R&D Systems)
⁶values represent mean fluorescence intensity staining of M21 cells bound with pooled serum and detected by fluorophore-conjugated anti-mIL12p40 antibody and analyzed by flow cytometry (control staining = 8)
n.d. not determined
n.a. not applicable

### Example 2

Induction of IFNγ Expression by RGD-mIL12 Fusion Protein.

IL12 activates NK and T cells, which respond by producing IFNγ. Maximum serum levels of IFNγ were observed 48-96 hours following HTV gene transfer of pNGVCmIL12 in mice (Lui 2002 and Table 1), showing that mIL12 produced following HTV gene delivery results in bioactive mIL12 that induces IFNγ synthesis in vivo.

Similarly, delivery of RGD/E mIL12 expression cassettes resulted in elevated IFNγ levels, indicating expression and secretion of active IL12 protein. Delivery of genes encoding chIL12 proteins failed to induce IFNγ production. IFNγ was detected in pooled serum samples 48 and 96 hr after gene delivery

These results indicated that all versions of the RGD-IL12 fusion protein expressed in vivo following gene delivery can be effectively detected and measured by mIL12p40 ELISA, and that fusion proteins containing mIL12 are biologically active and able to induce IFNγ synthesis.

### Example 3

Binding of RGD-IL12 Gene Product to αvβ3 Integrin.

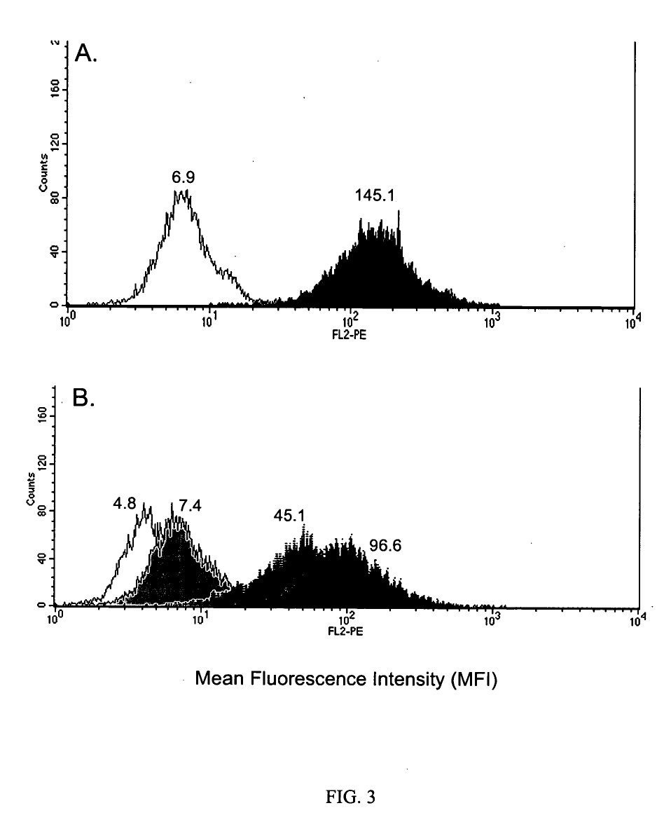

M21 human melanoma cells express high levels of surface αvβ3 integrins as shown by high level binding of anti-CD51/CD61 (i.e., anti-αvβ3 integrin: BD-PharMingen, CA) antibody (FIG. 3A) and a mean fluorescence intensity (MFI) of 145, with isotype control staining of only 6.9, as determined by flow cytometric analysis. In contrast, HeLa cervical carcinoma cells exhibit very low levels of αvβ3 integrin with an MFI of 8.3.

Using M21 and HeLa cells as αvβ3⁺ and αvβ3⁻ integrin expressing cells, respectively, we tested the ability of the pRGD-mIL12p40 and pRGD-IL12p35 in vivo expressed gene products to bind to these cells. M21 cells were pre-incubated with serum from ICR mice obtained 7 hr. following HTV delivery of 200 μg of control pDNA (pCMV-luciferase), pNGVC-mIL12, pRGD-mIL12p40, or pRGD-mIL12p35. Following washing, cell-bound RGD-IL12 fusion protein was detected by staining with anti-mIL12p70 fluorochrome-conjugated antibody (BD-PharMingen), which binds to the mIL12p70 (mp35/mp40) heterodimeric component of the RGD-IL12 molecule, and analyzed by flow cytometry. As shown in FIG. 3B, sera containing control pDNA or pNGVCmIL12 gene products exhibited minimal binding to M21 cells, with MFI values of 4.8 and 7.4, respectively. Conversely, the gene products expressed from pRGD-IL12p40 and pRGD-IL12p35 showed a high level of binding to M21 cells with MFI values of 45 and 97, respectively. Specificity of RGD-IL12 binding to the αvβ3 integrin was indicated by a lack of binding to HeLa cells, with MFI values between 4-8 for all pDNA gene products evaluated.

To evaluate binding of chIL12-containing RGD-IL12 gene products, anti-mIL12p40/p70 antibody was used. This detection antibody has specificity for the murine p40 subunit and the murine p70 heterodimer, and is analogous to the ELISA system that detects mIL12p40 serum levels. Pooled serum samples from ICR mice were evaluated for αvβ3 integrin binding for all versions of the RGD-IL12 gene products by testing the 12 hr time-point samples from mice that were injected with 20 μg pDNA. Table 1 shows the flow cytometry results of all individual gene products in binding to M21 cells. Differences in MFI levels between the data in FIG. 2 and Table 1 likely reflect the different antibody used in the analyses. Importantly, the anti-mIL12p40 detection method identified binding (MFI range 18-47) to the αvβ3 integrin of all fusion proteins that contain a functional RGD targeting ligand. In contrast, fusion proteins containing the nonfunctional RGE peptide component displayed no αvβ3 integrin-targeting (MFI=8). These results demonstrate that the in vivo gene products that possess the RGD peptide component effectively target and bind the αvβ3 integrin.

The data indicate that the RGD-IL12 fusion proteins expressed in vivo following HTV gene delivery of either pRGD-IL12(p40) or pRGD-IL12(p35) possess active IL12 components and are capable of binding to the αvβ3 integrin. In vivo delivery of pNGVCmIL12 results in expression of functional IL12 protein, but this molecule does not show binding specificity for the αvβ3 integrin.

### Example 4

Increased Intratumoral Levels of IL12 following RGD-IL12 Therapy.

To assess the potential of the RGD-IL 12 fusion protein to target the tumor microenvironment, we examined intradermal NXS2 tumors for mIL12-specific staining following HTV gene delivery. A/J mice bearing NXS2 i.d. tumors received 100 μg of pNGVCmIL12, pRGD-IL12p35, or Ringer's solution by HTV. Tumors were harvested 10 hr following HTV gene transfer. Harvested tumors were processed for mIL12 detection by immunohistochemistry. Harvested tumors were immersed in O.C.T. compound and snap-frozen in liquid nitrogen. 5-7 μm sections were prepared (Microm HM 505 N cryostat, Carl Zeiss, Goettingen, Germany), mounted on charged precleaned slides (Fisher Scientific) and air dried overnight at room temperature. The slides were fixed in 4% formaldehyde for 10 minutes, washed 3×3 minutes in PBS, and protein block (1% goat serum in PBS, 20 minutes) was applied. Slides were incubated with biotinylated goat anti-mouse IL-12 polyclonal antibody (Cell Science, Canton, Mass.), diluted 1:100, for 1.5 hrs at room temperature. After washing in PBS 3×3 minutes, the primary antibody was visualized with FITC-conjugated Strep-Avidin (4 mg/ml, dilution 1:250, 30 min). Slides were then stained for actin with Phalloidin-Alexa 488 (1:400) and for nuclear identification with To-Pro3 DNA (1:70,000) (both Molecular Probes, Eugene, Oreg.). All staining steps were performed in a humid chamber. Slides were examined using an LSM 510 confocal microscope (Zeiss, Germany). All images were scanned with identical settings under magnification X630.

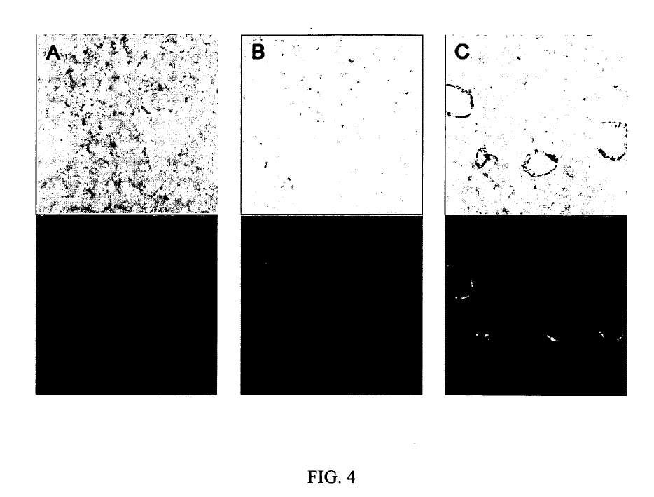

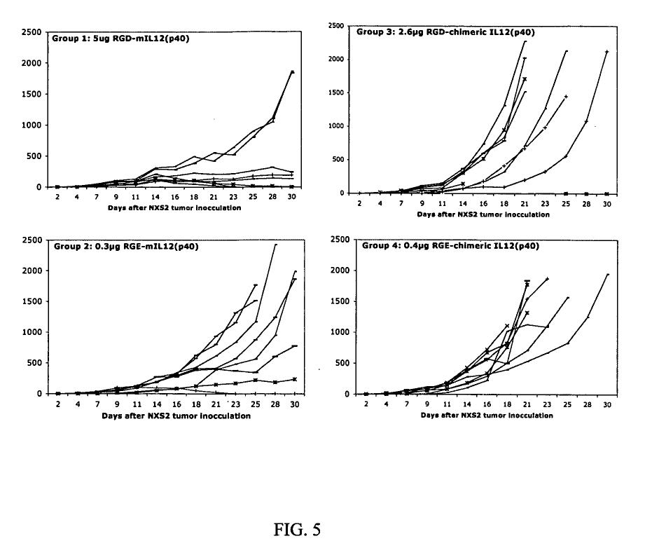

The images in FIG. 4 indicate that the tumor from an animal treated with pRGD-IL12p35 exhibited greater intratumoral staining for mIL12 than the tumor from a mouse that was treated with pNGVCmIL12. In FIG. 4, panels A, B, and C represent tumor sections from separate NXS2 tumor-bearing mice that received Ringer's solution, pNGVCmIL12, or pRGD-IL12p35, respectively. Upper frames are confocal images acquired for FITC emission, indicating staining for mIL12, while lower frames are composite images for nuclear (ToPro3, blue), actin (Phalloidin-Alexa 488, red), and mIL12 (green) staining. FIG. 4, panel C-upper indicates mIL12 staining of the luminal surface of several vascular structures within the tumor, indicating that the RGD-mIL12 fusion protein was bound to the tumor neovasculature. Although vascular structures are evident in the composite image (panel B-lower), similar mIL12 staining of the vascular lumen is absent in the tumor exposed to native mIL12 (panel B-upper). No intratumoral mIL12 staining was observed in the tumor from the mouse that received Ringer's solution (panel A).

### Example 5

RGD-IL12 Antitumor Response: In Vivo Antitumor Activity of the RGD-IL12 Molecule Against NXS2 i.d. Tumors.

The NXS2 tumor model has been shown to be susceptible to both antiangiogenic and IL12-mediated antitumor effects. A/J mice bearing measurable i.d. NXS2 tumors received 30, 10, 3, or 1 μg of pNGVCmIL12, pRGD-mIL12p40, or pRGD-mIL12p35 by HTV on day 12 following tumor inoculation, 4 mice per group. The combination of the tumor growth data from all four pDNA dose regimens showed that 9 of 15 tumors (60%) from pNGVCmIL12 treated mice resolved. Treatment with pRGD-IL12p40 resulted in complete regression of 12 of 14 (86%) established tumors. Treatment with pRGD-IL12p35 gene therapy induced tumor resolution of 8 of 13 (62%) tumors.

Additional antitumor efficacy studies were done in which the dose of pDNA was adjusted to express a predicted level of gene product based on the level of mIL12p40 detected in the serum following delivery of 5 μg of pRGD-mIL12p40. This standardization allows comparisons of gene therapies where similar levels of gene products are expressed in vivo. Groups (n=8) of NXS2 i.d. tumor-bearing mice received 5 μg of pRGD-mIL12p40, 0.3 μg of pRGE-mIL12p40, 2.6 μg of pRGD-chIL12p40, or 0.4 μg of RGE-chIL12p40 on day 8 following tumor engraftnent. As shown in FIG. 5, 6 of 8 pRGD-mIL12p40-treated mice (group 1) exhibited stable disease or successful tumor resolution (FIG. 5A). Only 2 of 8 mice that received pRGE-mIL12p40 had stable disease (FIG. 5B, group 2). Progressive tumor growth was observed in all mice that were given chIL12-containing gene products (FIG. 5C and D, groups 3 and 4), indicating that in the absence of a functional mIL12 effector moiety, the RGD-component (RGD-chIL12p40) had reduced antitumor effect. Group-pooled serum samples obtained 48 hr following HTV gene therapy indicated that test groups expressed similar levels of mIL12p40 (mIL12p40 values as determined by ELISA were 36, 29, 12, and 37 ng/ml for pRGD-mIL12p40-, pRGE-mIL12p40-, pRGD-chIL12p40- and pRGE-chIL12p40-treatement groups, respectively). IFNγ levels were similar between pRGD-mIL12p40 and pRGE-mIL12p40 treatment groups, with 7.3 and 7.0 ng/ml, respectively, and is suggestive that test groups were exposed to similar levels of mIL12-containing gene products in vivo. Groups that received chIL12-gene products exhibited low (<0.1 ng/ml) levels of IFNγ. These results indicate that when similar levels of mIL12 product is expressed in vivo, better antitumor responses are achieved with RGD-IL 12 as compared to non-targeted IL12 gene therapy.

Results indicate that delivered RGD-mIL12 fusion protein expression cassettes are expressed, bioactive, and secreted systemically into the blood vascular system (following HTV gene delivery). The RGD-IL12 fusion molecule binds to the αvβ3 integrin in vitro and demonstrates increased intratumoral IL12 levels in vivo. Most importantly, RGD-IL12 gene therapy shows enhanced antitumor effects when compared to non-targeted IL12 therapy.

### Example 6

Gene Delivery of Recombinant Chimeric Protein Expression Constructs.

Potential therapeutic chimeric proteins, such as the multifunctional RGD-IL12 fusion protein, can be tested for their effectiveness against appropriate tumors: (1) as prophylactic therapy to prevent tumor establishment, (2) as a therapeutic against smaller established tumors (tumor volume <75 mm³), and (3) as a therapeutic against larger established tumors (tumor volume >250 mm³). To test the prophylactic potential of chimeric protein gene therapy, the gene can be delivered to mice prior to establishment of the tumor, for example hepatic metastases. Tumor development or burden in mice expressing the chimeric protein is then compared with tumor development in mice expressing a control gene or untreated mice. Altering the day of initial gene delivery will aid in evaluating the effectiveness of the gene against different tumor progressions. Similarly, for testing the therapeutic benefit of gene delivery and in vivo expression of a chimeric protein on smaller and larger tumors, plasmid encoding the chimeric protein or control plasmid is delivered to mice have a given size tumor. Tumor progression is then monitored in both chimeric protein expressing and control mice. The appearance and level of soluble factors known to influence immunity and angiogenesis (such as IL12, IFNγ, and chemokines IP-10, MIG, and I-TAC) may also be measured. By testing different tumor models, including different types of tumors, the most effective chimeric protein to different tumors can be identified.

### Example 7

Intraportal Injections of Plasmid DNA:

After the livers of 25 g, 6-week old mice were exposed through a ventral midline incision, solutions containing pBS.CMVLux plasmid DNA (described below) were manually injected over approximately 30 sec into the portal vein using a 30-gauge, ½-inch needle and 1-ml syringe. In some animals, a 5×1 mm, Kleinert-Kutz microvessel clip (Edward Weck, Inc., Research Triangle Park, N.C.) was applied during the injection at the junction of the hepatic vein and caudal vena cava. Anesthesia was obtained from intramuscular injections of 1000 μg of ketamine-HCl (Parke-Davis, Morris Plains, N.J.) in 1 ml of normnal saline and methoxyflurane (Pitman-Moore, Mudelein, Ill. USA) which was administered by inhalation as needed. was purchased from Sigma. Heparin was purchased from LyphoMed (Chicago, Ill.).

Reporter Genes and Assays. The pBS.CMVLux, plasmid DNA was used to express luciferase from the human immediate early cytomegalovirus (CMV) promoter. At two days after injection, the livers were assayed for luciferase expression. The animals were sacrificed by cervical dislocation and the livers (average weight of 1.5 g) were divided into six sections composed of two pieces of median lobe, two pieces of left lateral lobe, the right lateral lobe, and the caudal lobe plus a small piece of right lateral lobe. Each of the six sections were placed separately into 200 μl of lysis buffer (0.1% Triton X-100, 0.1 M K-phosphate, 1 mM DTT pH 7.8) that was then homogenized using a homogenizer PRO 200 (PRO Scientific Inc., Monroe Conn.). The homogenates were centrifuged at 4,000 rpm for 10 min. at 4° C. and 20 μl of the supernatant were analyzed for luciferase activity. Relative light units (RLU) were converted to pg of luciferase using standards from Analytic Luminescence Laboratories (ALL, San Diego, Calif.). Luciferase protein (pg)=5.1×10⁻⁵×RLU+3.683 (r²=0.992). Total luciferase/liver was calculated by adding all the sections of each liver and multiplying by 23 to account for dilution effects. For each condition, the mean total luciferase/liver and the associated standard deviation are shown.

After the livers of 25 g, 6-week old mice were exposed through a ventral midline incision, 100 μg of pBS.CMVLux, plasmid DNA in 1 ml of solutions was injected into the portal vein via a 30-gauge, ½-inch needle over approximately 30 sec. Two days after injection, a mean of only 0.4 ng of total luciferase/liver was produced when the DNA was delivered intraportally in an isotonic solution without ligation of the hepatic vein (Table 1). Inclusion of 20% mannitol in the injection solution increased the mean total luciferase/liver over ten-fold to 4.8 ng (Table 1).

In order to prevent the DNA's rapid transit and to increase the intraportal hydrostatic pressure, the hepatic vein was clamped for two min after injection. Luciferase production increased another three fold to 14.7 ng (Table 1).

When the DNA was injected in a hypertonic solution containing 0.9% saline, 15% mannitol and 2.5 units/ml of heparin to prevent microvascular thrombosis and with the hepatic vein clamped, luciferase expression increased eight-fold to 120.3 ng/liver (Table 1). These results are also shown in Table 7 (no dexamethasone condition) in Example 3 below for each individual animal. If the mannitol was omitted under these conditions, luciferase expression was ten-fold less (Table 1).

These results indicate that hypertonicity, heparin and hepatic vein closure are required to achieve very high levels of luciferase expression.

Mean total luciferase in the liver following the intraportal injection (over 30 seconds) of 100 μg pBS.CMVLux in 1 ml of different solutions with no clamp or with the hepatic vein and inferior vena cava clamped for two minutes.

| | Mean | | |
|---|---|---|---|
| | Luciferase | Standard | Number of |
| Condition | (total ng/liver) | Error | Livers |
| no clamp, normal saline solution | 0.4 | 0.7 | n = 6 |
| (NSS) | | | |
| no clamp, 20% mannitol | 4.8 | 8.1 | n = 3 |
| clamp, 20% mannitol | 14.6 | 26.3 | n = 9 |
| clamp, 2.5 units heparin/ml in | 11.8 | 12.5 | n = 4 |
| NSS | | | |
| clamp, 15% mannitol and 2.5 | 120.3 | 101.5 | n = 12 |
| units heparin/ml in NSS | | | |

Luciferase activities in each liver were evenly distributed in six divided sections assayed (Table 2). All six parts of each liver from all three animals had substantial amounts of luciferase. This is in marked contrast to the direct interstitial, intralobar injection of DNA in which the expression is restricted to the site of injection.

The distribution of luciferase expression over the six liver sections in animals injected intraportally (over 30 seconds) with 100 μg of pBS.CMVLux in 1 ml of normal saline solution plus 15% mannitol and 2.5 units heparin/ml and with the hepatic vein clamped for 2 minutes.

| | Total luciferase/Liver | | |
|---|---|---|---|
| | (ng/Liver/mouse) | | |
| Liver Section | Mouse #1 | Mouse #2 | Mouse #3 |
| ½ of median lobe | 496.5 | 66.9 | 304.5 |
| other ½ of median | 177.0 | 126.1 | 241.4 |
| lobe | | | |
| ½ of left lateral | 763.8 | 208.7 | 325.2 |
| lobe | | | |
| other ½ of left | 409.4 | 160.4 | 218.9 |
| lateral lobe | | | |
| right lateral lobe | 527.8 | 129.7 | 216.2 |
| caudal lobe + small | 374.1 | 149.7 | 240.8 |
| piece of right lateral | | | |
| lobe | | | |
| Total | 2,748.6 | 841.5 | 1,547.0 |
| Mean | 458.1 | 140.3 | 257.8 |
| Range | 177-763 | 67-209 | 216-325 |
| Standard Deviation | 194.0 | 46.6 | 45.9 |

Conclusions:
1. High levels of luciferase expression were obtained from injecting 100 μg of pBS.CMVLux intraportally.
2. The highest levels of luciferase expression were obtained when the animals were injected intraportally over 30 seconds with 100 μg of pBS.CMVLux in 1 ml of normal saline solution plus 15% mannitol and 2.5 units heparin/ml and with the hepatic vein clamped for 2 minutes.
3. These high levels of expression were consistently obtained in dozens of mice.
4. The luciferase expression was evenly distributed throughout the liver.

### Example 8

The Effects of other Factors on Expression following Intraportal Injection of pBS.CCMVLux.

Unless otherwise specified, the intraportal injections and luciferase assays were done as above.

Effect of time of hepatic vein occlusion on luciferase expression in animals injected intraportally with 100 μg of pBS.CMVLux in 1 ml of normal saline solution plus 15% mannitol and 2.5 units heparin/mi. The times for which the hepatic vein was occluded were varied from 2 min to 4 min and to 6 min. The time of occlusion did not have a large effect on expression.

| | Total luciferase/Liver | | |
|---|---|---|---|
| | (ng/Liver/mouse) | | |
| Mouse Number | 2 min | 4 min | 6 min |
| 1 | 4.6 | 1.9 | 32.7 |
| 2 | 44.9 | 11.5 | 6.4 |

Effect of length of injection (time it took to inject all of the 1 ml) on luciferase expression in animals injected intraportally with 100 μg of pBS.CMVLux in 1 ml of normal saline solution plus 15% mannitol and 2.5 units heparin/ml and with the hepatic vein occluded for 2 min. The times over which the injections were done were varied from 30 seconds to 1 minute and 2 minutes.Injecting the 1 ml of the DNA solution (100 μg pBS.CMVLux) over 30 seconds enabled the highest levels of luciferase expression. Longer times of injection led to lower levels.

| | Total luciferase/Liver | | |
|---|---|---|---|
| | (ng/Liver/mouse) | | |
| Mouse Number | 30 sec | 1 min | 2 min |
| 1 | 2,697 | 188 | 21.6 |
| 2 | 790 | 13.4 | 19.9 |
| 3 | 1,496 | 141.1 | 11.8 |
| Mean | 1,662 | 114 | 18 |
| Standard Deviation | 964 | 91 | 5 |

Total luciferase expression in each liver of each animal injected intraportally (over 30 sec) with 100 μg of pBS.CMVLux in either 0.5 or 1 ml of normal saline solution plus 15% mannitol and 2.5 units heparin/ml and with the hepatic vein occluded for 2 min. If the total volume of the injection fluid was 0.5 ml instead of 1.0 ml, luciferase expression decreased 70-fold.

Total luciferase/
Liver
(ng/Liver/mouse)

| Mouse Number | 0.5 ml | 1 ml |
|---|---|---|
| 1 | 1.6 | 51.9 |
| 2 | 4.7 | 124.8 |
| 3 | 0.4 | 266.9 |
| Mean | 2.3 | 147.9 |
| Standard | 2.3 | 109.4 |
| Deviation | | |

Conclusions:
1. The optimal conditions are in fact the conditions first described in example 1: the animals were injected intraportally over 30 seconds with 100 μg of pBS.CMVLux in 1 ml of normal saline solution plus 15% mannitol and 2.5 units heparin/ml and with the hepatic vein clamped for 2 minutes.
2. Use of 500 μg of pBS.CMVLux did not enable greater levels of expression but expression was approximately7-fold less if 20 μg of DNA was used.
3. Occluding the hepatic vein for longer than 2 minutes did not increase expression.
4. Injecting the pBS.CMVLux over 30 seconds gave the highest luciferase levels as compared to injection times longer than 30 seconds.
5. Injecting the pBS.CMVLux in 1 ml gave higher luciferase levels than injecting the pBS.CMVLux in 0.5 ml.

### Example 9

Delivery to Spleen.

After the portal veins of 25 g, 6-week old mice were exposed through a ventral midline incision, 100 μg of pBS.CMVLux plasmid DNA in 0.5 ml or 1 ml of normal saline solution plus 15% mannitol and 2.5 units heparin/ml were manually injected over 30 seconds into the portal vein near the junction of the splenic vein and portal vein. The portal vein had two clamps placed distal and proximal to the point of injection so as to direct the injection fluid into only the splenic vein and to prevent the injection fluid from going to the liver or intestines. The injections were done using a 30-gauge, ½-inch needle and 1-ml syringe. 5×1 mm, Kleinert-Kutz microvessel clips (Edward Weck, Inc., Research Triangle Park, N.C.) were used. Anesthesia was obtained from intramuscular injections of 1000 μg of ketamine-HCI (Parke-Davis, Morris Plains, N.J.) and methoxyflurane (Pitman-Moore, Mudelein, Ill. USA) which was administered by inhalation as needed. was purchased from Sigma. Heparin was purchased from LyphoMed (Chicago, Ill.). Two days after injection the spleens and pancreas were removed and placed in 500 μl of lysis buffer and 20 μl were analyzed for luciferase expression as described above.

Luciferase expression after the intravascular-administration of pBS.CMVLux into the splenic vein via the portal vein. Substantial amounts of luciferase activity were obtained in the spleen and pancreas of all four mice with both injection fluids of 0.5 ml and 1 ml.

Total luciferase/Organ (pg/organ/mouse)

| Injection Volume | Spleen | Pancreas |
|---|---|---|
| 0.5 ml | 814.4 | 97.2 |
| 0.5 ml | 237.3 | 88.7 |
| 1 ml | 168.7 | 109.4 |
| 1 ml | 395.0 | 97.7 |
| Mean | 403.9 | 98.3 |
| Standard | 289.6 | 8.5 |
| Deviation | | |

### Example 10

Delivery of Transgene to Skeletal Muscle.

100 μg of pBS.CMVLux in 10 ml of normal saline solution plus 15% mannitol was injected into the femoral artery of adult rats with the femoral vein clamped. One to four days after injection, the quadricep was removed and cut into 10 equal sections. Each sections were placed into 500 μl of lysis buffer and 20 μl were assayed for luciferase activity as described above.

Luciferase expression in the quadricep of a rat after the injection of 100 μg of pBS.CMVLux into the femoral artery and with the femoral vein clamped. Substantial amounts of luciferase expression were expressed in the quadriceps following the intravascular delivery of plasmid DNA. Intravascularly-administered plasmid DNA can express efficiently in muscle.

| | Total Luciferase |
|---|---|
| Rat Number | (pg/quadriceps) |
| 1 | 157.5 |
| 2 | 108.8 |
| 3 | 139.2 |
| 4 | 111.3 |
| Mean | 129.2 |
| Standard Deviation | 23.4 |

### Example 11

Retrograde Injection into Efferent Vessel of Target Tissue.

In the liver, the hepatic vein is an efferent blood vessel since it normally carries blood away from the liver into the inferior vena cava. The portal vein, hepatic arteries, and tail vein are afferent blood vessels in relation to the liver since they normally carry blood towards the liver. 100 μg of pCILuc in 1 ml of normal saline solution plus 15% mannitol and 2.5 units heparin/ml were injected over 30 seconds into hepatic vein via the inferior vena cava. Since it was difficult to directly inject the hepatic vein in rodents, the injections were directed into the inferior cava which was clamped in two locations; proximal and distal (i.e. downstream and upstream) to the entry of the hepatic vein into the inferior vena cava. Specifically, the downstream inferior vena cava clamp was placed between the diaphragm and the entry point of the hepatic vein. The upstream inferior vena cava clamp was placed just downstream of the entry point of the renal veins. Therefore, the 1 ml of the injection fluid entered the hepatic vein and the liver. Since the veins of other organs such as the renal veins enter the inferior vena cava at this location, not all of the 1 ml of injection fluid goes into the liver.

In some of the animals that received retrograde injections in the inferior vena cava, the hepatic artery, mesenteric artery, and portal vein were clamped (occluded) for approximately five minutes immediately before and then after the injections. Specifically, the order of placing the clamps were as follows: first on hepatic artery, then portal vein, then downstream vena cava, and then upstream vena cava. It took about three minutes to place all these clamps and then the injections were done. The clamps were left in place for an additional two minutes from the time that the last clamp (upstream vena cava clamp) was placed.

The intraportal injections were performed as stated using optimal intraportal injections over 30 seconds with 100 μg of pCILuc in 1 ml of normal saline solution plus 15% mannitol and 2.5 units heparin/ml and with the hepatic vein clamped for 2 minutes. Some of the mice also received daily subcutaneous injections of 1 mg/kg of dexamethasone (Elkins-Sinn, Cherry Hill, N.J.) starting one day prior to surgery. The pCILuc plasmid expresses a cytoplasmic luciferase from the CMV promoter.

Two days after the injections, the luciferase activity was measured as above in six liver sections composed of two pieces of median lobe, two pieces of left lateral lobe, the right lateral lobe, and the caudal lobe plus a small piece of right lateral lobe. Inferior Vena Cava/Hepatic Vein Injections with the Portal Vein and Hepatic Artery Clamped (*Injections in animal #3 were not optimal since the fluid leaked during the injections.) Injections were done in 6-week old animals that received dexamethasone.

| | Luciferase Activity (ng) | | |
|---|---|---|---|
| Sections | Animal #1 | Animal #2 | Animal #3* |
| 1 | 5,576.7 | 4,326.4 | 1,527.4 |
| 2 | 8,511.4 | 4,604.2 | 1,531.6 |
| 3 | 5,991.3 | 5,566.1 | 2,121.5 |
| 4 | 6,530.4 | 9,349.8 | 1,806.3 |
| 5 | 8,977.2 | 4,260.1 | 484.2 |
| 6 | 9,668.6 | 6,100.2 | 1,139.3 |
| total liver | 45,255.5 | 34,206.9 | 8,610.4 |
| mean | 29,357.6 | | |
| standard deviation | 18,797.7 | | |

Inferior Vena Cava/Hepatic Vein Injections with the Portal Vein and Hepatic Artery not Clamped. Injections were done in 6-week old animals that did not receive dexamethasone.

Luciferase Activity (ng)

| Sections | Animal #1 | Animal #2 |
|---|---|---|
| 1 | 360.6 | 506.2 |
| 2 | 413.5 | 724.7 |
| 3 | 463.0 | 626.0 |
| 4 | 515.5 | 758.6 |
| 5 | 351.6 | 664.8 |
| 6 | 437.8 | 749.6 |
| total liver | 2,542.0 | 4,029.8 |
| mean | 3,285.9 | |
| standard deviation | 1,052.1 | |

Portal Vein Injections with the Hepatic Vein Clamped in 6 month old mice that received dexamethasone.

| | Luciferase Activity (ng) | | |
|---|---|---|---|
| Sections | Animal #1 | Animal #2 | Animal #3 |
| 1 | 287.4 | 417.0 | 129.2 |
| 2 | 633.7 | 808.1 | 220.5 |
| 3 | 689.8 | 1,096.5 | 328.2 |
| 4 | 957.8 | 1,056.9 | 181.6 |
| 5 | 660.7 | 1,487.4 | 178.6 |
| 6 | 812.4 | 1,276.4 | 233.4 |
| total liver | 4,041.8 | 6,142.2 | 1,271.5 |
| mean | 3,818.5 | | |
| standard deviation | 2,443.0 | | |

Portal Vein Injections with the Hepatic Vein Clamped in 6 week old mice that received dexamethasone.

| | Luciferase Activity (ng) | | |
|---|---|---|---|
| Sections | Animal #1 | Animal #2 | Animal #3 |
| 1 | 352.9 | 379.1 | 87.0 |
| 2 | 667.5 | 373.9 | 108.2 |
| 3 | 424.8 | 1,277.9 | 178.4 |
| 4 | 496.3 | 1,308.6 | 111.9 |
| 5 | 375.2 | 296.4 | 162.3 |
| 6 | 434.7 | 628.7 | 123.0 |
| total liver | 2,751.4 | 4,264.7 | 770.9 |
| mean | 2,595.7 | | |
| standard deviation | 1,752.1 | | |

Conclusions:
1. Retrograde delivery of plasmid DNA into the efferent vessels of the liver via the hepatic vein/inferior vena cava leads to high levels of gene expression.
2. The highest levels were achieved using this retrograde approach if the afferent vessels to the liver (portal vein and hepatic artery) were occluded.
3. Under all conditions, luciferase expression was evenly distributed throughout all six liver sections.

The foregoing is considered as illustrative only of the principles of the invention. Furthermore, since numerous modifications and changes will readily occur to those skilled in the art, it is not desired to limit the invention to the exact construction and operation shown and described. Therefore, all suitable modifications and equivalents fall within the scope of the invention.

1. A process for treating cancer in a mammal comprising:
a) forming a nucleic acid expression vector encoding a therapeutic chimeric protein comprising an effector subunit and a targeting subunit;
b) inserting said vector into a vessel in said mammal thereby delivering said vector to extravascular cells in said mammal; and
c) expressing said protein.

2. The process of claim 1 wherein said protein is secreted into the circulation of said mammal.

3. The process of claim 1 wherein said targeting subunit has affinity for receptors on tumor cells, cells associated with a tumor, cells necessary for tumor growth, or cells known to have antitumor activity.

4. The process of claim 3 wherein said targeting subunit increases a therapeutic index of said effector subunit.

5. The process of claim 3 wherein said targeting subunit has affinity for integrin receptors.

6. The process of claim 5 wherein said targeting subunit comprises an RGD targeting moiety.

7. The process of claim 1 wherein said effector subunit comprises interleukin 12 or a functional fragment of interleukin 12.

8. The process of claim 1 wherein said targeting subunit possesses antitumor activity.

9. The process of claim 2 wherein the vector is delivered to a liver cell.

10. The process of claim 2 wherein the vector is delivered to a muscle cell.

11. The process of claim I wherein the vector consists of a naked polynucleotide.

12. The process of claim 1 wherein said vector is associated with a non-viral complex.

13. The process of claim 1 wherein the cancer consists of a vascularized tumor.

14. A compound for treating ovarian cancer comprising a targeting moiety linked to an effector subunit having antitumor activity.

15. The compound of claim 14 wherein the targeting moiety comprises a peptide, protein subunit, protein fragment, or full-length protein that binds to ovarian tumor cells through interaction with cell surface molecules.

16. The compound of claim 15 wherein the targeting moiety is selected from the group consisting of: Anti-Müllerian hormone and AMH-receptor ligands.

17. The compound of claim 14 wherein the effector subunit comprises interleukin 12 or a functional fragment of interleukin 12.

18. The compound of claim 14 wherein the compound is encoded by a nucleic acid sequence.

19. The compound of claim 18 wherein the compound is produced in vivo following delivery of the nucleic acid sequence to a cell in a mammal.


SEQUENCE LISTING (1 sequence)

SEQ ID NO: 1 - protein, 9 aa, Artificial
  synthetic integrin binding peptide
CDCRGDCFC